(19) United States
(12) Patent Application Publication
(10) Pub. No.: US 2009/0145439 A1
(43) Pub. Date: Jun. 11, 2009

(54) Medical tube holder

(76) Inventor: Cory William Peichel, Richardson, TX (US)

Correspondence Address: Cory W. Peichel, 1816 Auburn Drive, Richardson, TX, 75081, US

(21) Appl. No.: 11/999,825

(22) Filed: Dec. 8, 2007

(51) Int. Cl. A61M 25/02

(52) U.S. Cl. 128/207.17

(57) ABSTRACT

A medical tube holder that is designed to secure a medical tube, such as a standard tracheostomy tube. In one embodiment the medical tube holder includes: (1) a single-piece, nonabsorbent retention band with a length based on the circumference of a patient's neck and (2) two hook fastener portions, which can be looped through slots on a medical tube flange and connected back onto a loop fastener piece that is attached to the nonabsorbent retention band.

### CROSS-REFERENCE TO RELATED APPLICATION

This application is based on, and claims priority of, U.S. Provisional Patent Application Ser. No. 60/861,831, filed by Peichel on Dec. 20, 2006, entitled “Medical Tube Holder,” commonly owned herewith and incorporated herein by reference.

### TECHNICAL FIELD OF THE INVENTION

The present invention is directed in general to medical tube holders and more particularly to tracheostomy tube holders that can be used to effectively support and secure a tracheostomy tube, preventing it from rotating, pivoting, migrating, or completely dislodging.

### BACKGROUND OF THE INVENTION

A tracheotomy is a fairly common surgical procedure that is performed by cutting through the front of the neck and trachea to facilitate respiration, which can be impaired due to a tracheal or laryngeal defect or due to an obstruction. A tracheostomy tube is inserted through the skin and into the trachea to keep the airway patent, but because the tube can dislodge without support, it must be held in place by some kind of tie or tube holder.

Almost all tracheostomy tubes are manufactured with a flange, also known as a neck plate, which extends laterally from the proximal end of the tube running parallel to the front of a patient's neck. The neck plate limits the depth of penetration of the tracheostomy tube and also limits the movement of the tube relative to the plate. To keep the flange snug against the neck, it is manufactured with two slots through which a tracheostomy tube holder is threaded. The tracheostomy tube holder is looped around the patient's neck and tied or fastened to hold the tracheostomy tube in place.

Rolls of twill tape or thin strips of cloth can be used to secure the position of a tracheostomy tube. To keep the tube from migrating a portion is cut from the roll, wrapped snug around the patient's neck, slipped through the two slots in the flange, and tied into a knot to hold the tracheostomy tube in position. The twill tape when used as described creates several problems. The tape irritates the patient's neck, and the knot often rubs and breaks down the skin. Not only is the knot difficult and time consuming to place, but it can come undone if not tied properly. Finally, because the tape is made of cloth, the material soils and harbors bacteria and other microorganisms that can cause disease.

Wapner discloses a tracheostomy tube holder in U.S. Pat. No. 4,331,144 in which he uses a band that is composed of two straps to encircle the neck. This device uses narrow hook and loop fastener tabs to thread through the flange slots and then loop back to secure to the cotton-lined pile on the neckband. It also uses a hook and loop fastener to connect the two straps, which form the length of available band. Dale Medical Products Inc., of Plainville, Mass. currently produces a commercial product, The Dale 240 Tracheostomy Tube Holder, based on Wapner's design.

Disadvantages of using a device derived from '144 or any medical tube holder made from cloth or open cell foam include a relatively high cost for each unit and a significant reoccurring cost because of the need to replace the device frequently. This tracheostomy tube holder, like the twill tape, soils easily and absorbs saliva, sweat, or water from a bath or shower. Also, the device is bulky and warm and if any of the fastener hooks are not centered properly on the band, it will scratch the patient's neck.

Another tracheostomy tube holder on the market is a relatively simple metal chain. Transtracheal of Englewood, Colo. produces and sells an EZ-Chain Catheter Necklace that is manufactured of sterling silver and is approximately twenty inches long. The EZ-Chain combines a lobster claw clasp for length adjustment, a fold over clasp for easy chain removal, and a magnetic clasp for additional security. One advantage of using a chain is that it will not soil like a cloth tracheostomy tube holder and can be left in place during a bath. The biggest disadvantage of using a chain is that it is extremely difficult to get on and off the patient. If chains are used, caregivers must keep wire cutters handy in case of an emergency. Also, a chain can fatigue and break at a link or near the clasp. When that happens, the tracheostomy tube will almost always dislodge.

To summarize, tracheostomy tube holders heretofore known suffer from a number of disadvantages which include:

(a) Those made from cloth or open cell foam are easily soiled and absorb water, secretions, or sweat to create an environment that harbors disease-causing microorganisms.

(b) Those made from cloth or open cell foam must be changed after a bath or shower because they stay wet and have a potential to shred.

(c) Those made from cloth or open cell foam are often very wide and are too warm during summer months.

(d) Those made from cloth or open cell foam are bulky and cause the wearer to stand out as a patient, especially in a non-clinical setting.

(e) The hooks on those made from cloth or open cell foam scratch the neck if the tab is not centered properly.

(f) Those made from a metal chain are difficult to size and difficult to cut to the correct length.

(g) Those made from a metal chain are difficult to get on and off.

(h) Those made from a metal chain require a special tool, such as a wire cutter, to be immediately available.

(i) Those made from a metal chain appear secure, but wire and clasps fatigue and break unexpectedly.

Accordingly, what is needed in the art is a tracheostomy tube holder that combines the advantages of a cloth device with the advantages of a metal device, while minimizing the disadvantages. This new tracheostomy tube holder needs to be comfortable, needs to stay sanitary, needs to be easy to get on and off, needs to hold the tracheostomy tube securely, and needs to allow the patient to wash or to be washed with the ties in place.

### SUMMARY OF THE INVENTION

To address the above-discussed deficiencies of the prior art, the present invention provides a medical tube holder.

In one embodiment the medical tube holder includes: (1) a single-piece, nonabsorbent retention band with a length based on the circumference of a patient's neck and (2) two hook fastener portions, which can be looped through slots on a medical tube flange and connected back onto a loop fastener piece that is attached to the nonabsorbent retention band.

In another embodiment the medical tube holder includes: (1) a three-piece, nonabsorbent retention band that can be adjusted to a length based on the circumference of a patient's neck and (2) two hook fastener portions, which can be looped through slots on a medical tube flange and connected back onto a loop fastener piece that is attached to the nonabsorbent retention band.

In another embodiment the medical tube holder includes: (1) a single-piece, nonabsorbent retention band with a length based on the circumference of a patient's neck and (2) two hook fastener portions, which can be looped through slots on a medical tube flange and connected back onto two loop fastener pieces that are attached to the nonabsorbent retention band.

The foregoing has outlined preferred and alternative features of the present invention so that those skilled in the pertinent art may better understand the detailed description of the invention that follows. Additional features of the invention will be described hereinafter that form the subject of the claims of the invention. Those skilled in the pertinent art should appreciate that they can readily use the disclosed conception and specific embodiment as a basis for designing or modifying other structures for carrying out the same purposes of the present invention. Those skilled in the pertinent art should also realize that such equivalent constructions do not depart from the spirit and scope of the invention.

### BRIEF DESCRIPTION OF THE DRAWINGS

For a more complete understanding of the invention, reference is now made to the following descriptions taken in conjunction with the accompanying drawings, in which:

FIG. 1 is a front perspective view of one embodiment of a medical tube holder constructed according to the principles of the present invention that includes a nonabsorbent retention band and two fastener tabs that are wrapped through the slots of a flange of a medical tube;

FIG. 2-A is a side view of the embodiment of FIG. 1 and shows the side positioned farthest away from the patient that includes the nonabsorbent retention band and the two fastener tabs;

FIG. 2-B is an expanded side view of the embodiment of FIG. 2-A that shows a closed-cell polymer foam piece situated near the patient, a loop fastener piece situated away from the patient, and a hook fastener piece situated between the closed-cell polymer foam piece and the loop fastener piece;

FIG. 3 is a front perspective view of an alternative embodiment of a medical tube holder constructed according to the principles of the present invention that includes a three-piece, nonabsorbent retention band and two fastener tabs that are wrapped through the slots of a flange of a medical tube;

FIG. 4-A is a disassembled side view of the embodiment of FIG. 3 that shows two interlocking pieces of the nonabsorbent retention band and a loop fastener piece;

FIG. 4-B is a partially-assembled side view of the embodiment of FIG. 4-A that shows the two interlocking pieces of the nonabsorbent retention band and the loop fastener piece;

FIG. 4-C is a partially-assembled top view of the embodiment of FIG. 4-B that shows the two interlocking pieces of the nonabsorbent retention band and the loop fastener piece;

FIG. 5 is a front perspective view of an alternative embodiment of a medical tube holder constructed according to the principles of the present invention that includes a nonabsorbent retention band and two fastener tabs that are wrapped through the slots of a flange of a medical tube;

FIG. 6-A is a side view of the embodiment of FIG. 5 that shows the side positioned farthest away from the patient that includes the nonabsorbent retention band and the two fastener tabs;

FIG. 6-B is an expanded side view of the embodiment of FIG. 6-A that shows a neoprene piece situated near the patient, two loop fastener pieces situated away from the patient, and two hook fastener pieces situated between the neoprene piece and the loop fastener pieces.

### DETAILED DESCRIPTION

The present invention provides a medical tube holder that can be used to comfortably secure a medical tube, such as a tracheostomy tube, to a patient's neck. In each embodiment the medical tube holder is easy to get on and off, stays hygienic, is nonabsorbent, and dries easily after a bath or shower.

Because most current medical tube holders are made from cloth or open cell foam, they have a tendency to absorb sweat, stoma secretions, and any other liquid with which they come in contact. If a patient takes a shower or a bath, the device often saturates with water. Because these medical tube holders take so long to dry, they can cause skin irritation and breakdown. Also, after as little as a day, the band on this type of medical tube holder begins to curl and fray, which weakens its ability to hold the tracheostomy tube securely.

By substituting the cotton stretch material, open cell foam, or metal chain of today's medical tube holders with a nonabsorbent solid, woven, or closed-cell polymer foam material such as natural or synthetic rubber, neoprene, polypropylene, polyethylene, polyvinyl chloride, or nylon, problems with absorption, degradation, and staining are reduced, and the safety of the device is greatly improved.

A medical tube holder that uses a nonabsorbent material in the retention band is engineered to be pliable and is molded smooth to promote comfort. Because the retention band is made with a nonabsorbent material, the patient can be washed with the medical tube holder in place and then toweled dry. There is no longer a need to change the device after each wash. Furthermore, by keeping the width of the retention band fairly narrow, the insulating effect of the device is minimized, which keeps the patient cool even during the warmest summer months.

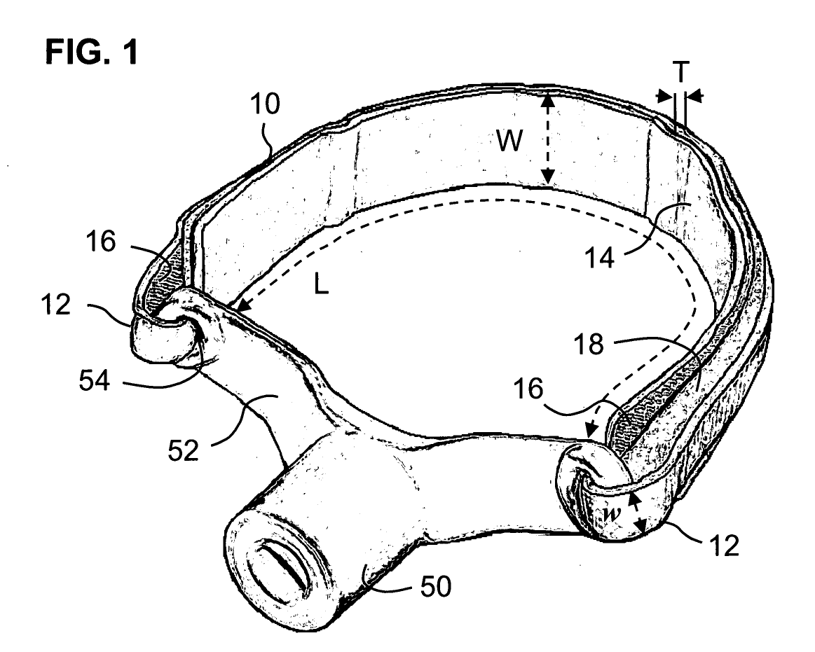

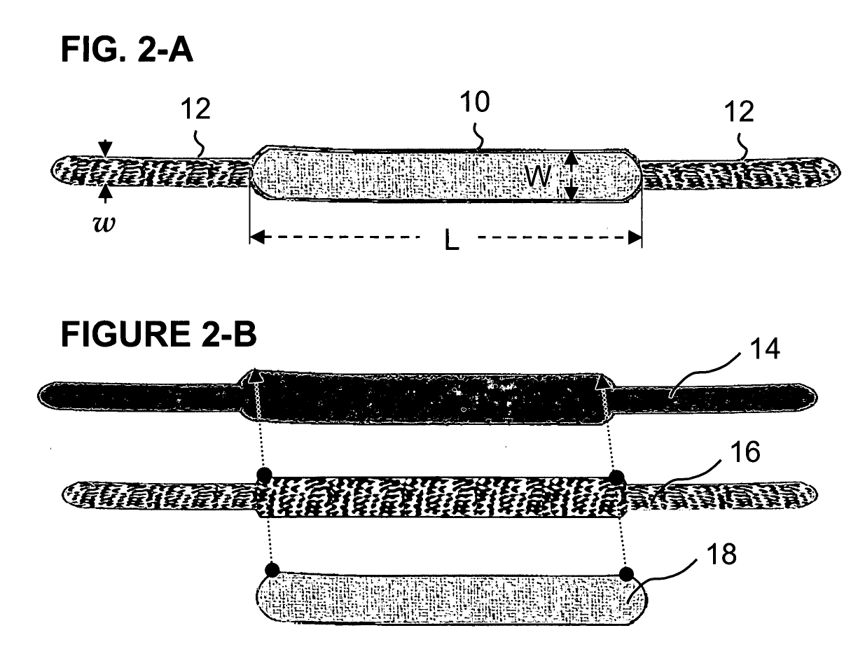

The primary embodiment of the present invention will now be described. A medical tube holder is illustrated in FIG. 1 (perspective view), FIG. 2-A (side view), and FIG. 2-B (side view). The medical tube holder includes a nonabsorbent retention band 10 with two fastener tabs 12 wrapped through the slots 54 of a flange 52 of a medical tube 50.

The dimensions of the medical tube holder are approximate and can vary from device to device. The width W of the nonabsorbent retention band 10 is approximately fifteen millimeters, much less than today's commonly used medical tube holders. The width w of the two fastener tabs 12 is sized at approximately four millimeters to fit through the slots 54 of the medical tube 50. The thickness T of the nonabsorbent retention band 10 is approximately four millimeters. The length L of the nonabsorbent retention band 10 is sized roughly one flange-length less than the circumference of the prospective patient's neck.

At each end of the medical tube holder, a fastener tab 12 extends out from the nonabsorbent retention band 10. The length of each fastener tab 12 is up to fifty percent of the length L of the nonabsorbent retention band 10. This allows the medical tube holder to be used across a range of patients and prevents the fastener tabs 12 from overlapping when applied.

A portion of a hook fastener piece 16 is coupled to each fastener tab 12. The fastener tabs 12 secure the medical tube 50 by slipping a portion of each through a slot 54 on the medical tube flange 52 and connecting back to a loop fastener piece 18 that is coupled to the nonabsorbent retention band 10.

A closed-cell polymer foam piece 14, which is situated closest to the patient, is used to provide comfort and prevent the absorption of moisture. A hook fastener piece 16 is secured to the closed-cell polymer foam piece 14 using an adhesive. The hook fastener piece 16 is shaped similarly to the closed-cell polymer foam piece 14 but is set in from the edges. A double-sided loop fastener piece 18 is secured to the wider portion of the hook fastener piece 16 using one side and connects to the fastener tabs 12 using the opposite side.

Closed-cell polymer foam is not the only material that can be used as the nonabsorbent material and can be substituted with a relatively flexible, nonabsorbent material. Several varieties of material are acceptable including a nonabsorbent solid, woven plastic braid, or closed-cell foam material made from natural or synthetic rubber, neoprene, polypropylene, polyethylene, polyvinyl chloride, or nylon. Any of these or a like material is acceptable as long as it has the flexibility and resilience necessary to prevent the absorption of moisture, to hold the medical tube 50 securely, and to remain hygienic.

Also, one of several different fasteners, including a hook and loop fastener similar to that used on today's tracheostomy tube holders, can be used to connect the medical tube holder to the flange 52 of the medical tube 50. A low profile mushroom stemmed fastener, a tongue and groove fastener, or a similar fastener can also be used because any of these can be opened and closed repeatedly to allow the caregiver to periodically change the tracheostomy tube. Any of these fasteners can be engineered to be highly pliable and still maintain a very secure hold.

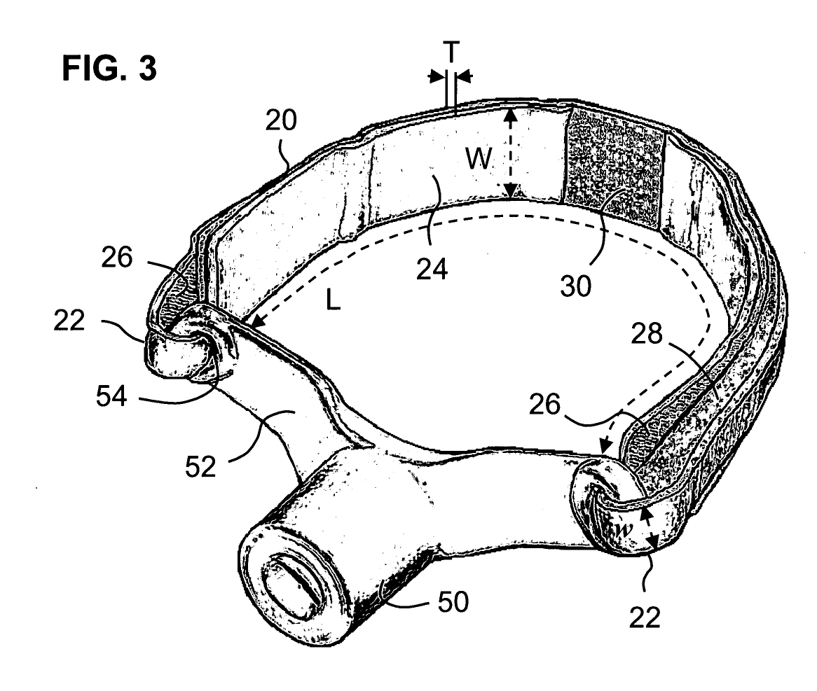

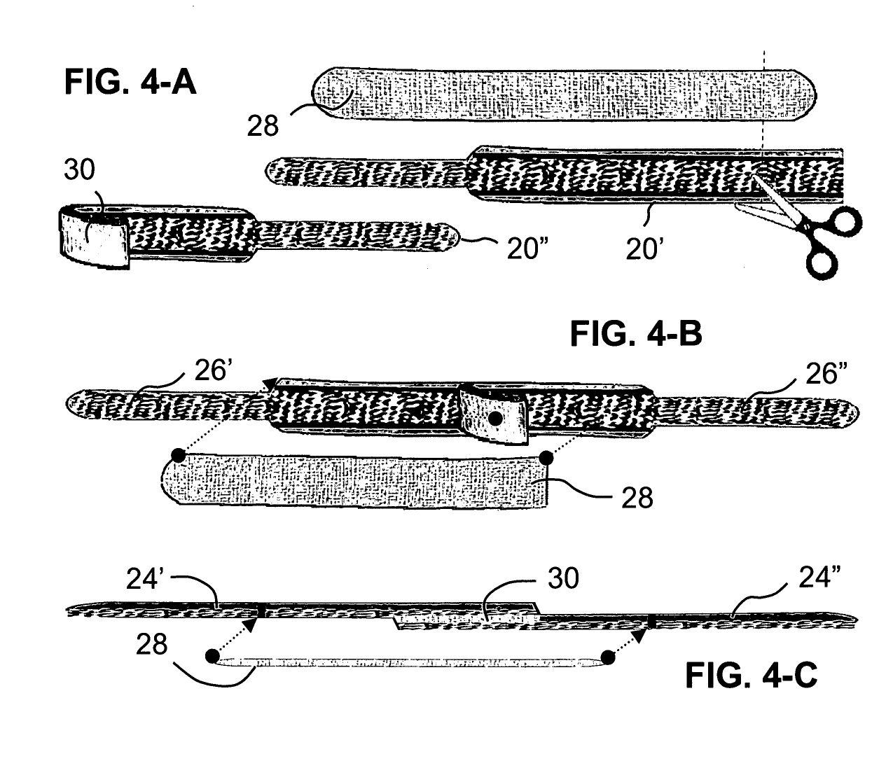

An alternative embodiment of the present invention is illustrated in FIG. 3 (perspective view), FIG. 3-A (side view), FIG. 3-B (side view), and FIG. 3-B (top view). The medical tube holder is constructed in a manner similar to that of the primary embodiment and includes a modified nonabsorbent retention band 20 with two fastener tabs 22 wrapped through the slots 54 of a flange 52 of a medical tube 50.

The physical dimensions (W, w, T, L) of the alternative embodiment are sized similarly to those of the primary embodiment. Also similar to the primary embodiment, the fastener tabs 22 are up to fifty percent of the length L of the nonabsorbent retention band 20 and are used in a similar manner to secure the medical tube 50.

For the alternative embodiment the nonabsorbent retention band 20 is comprised of two interlocking pieces 20′ and 20″. The interlocking pieces 20′ and 20″ are comprised of two closed-cell polymer foam pieces 24′ and 24″ and two hook fastener pieces 26′ and 26″. The hook fastener pieces 26′ and 26″ are secured to the closed-cell polymer foam pieces 24′ and 24″ using an adhesive. The hook fastener pieces 26′ and 26″ are shaped similarly to the closed-cell polymer foam piece 24′ and 24″ but are set in from the edges.

One of the interlocking pieces of nonabsorbent retention band 20′ can be adjusted for length by cutting away a measured piece. The other piece of nonabsorbent retention band 20″ cannot be adjusted for length because it incorporates a small loop fastener tab 30 at one end, which slides over and connects to the first piece of nonabsorbent retention band 20′.

A double-sided loop fastener piece 28 can also be adjusted for length by cutting away a measured piece. After the interlocking pieces of nonabsorbent retention band 20′ and 20″ are connected, the loop fastener piece 28 is secured to the wider portion of the hook fastener pieces 26′ and 26″ using one side and connects to the fastener tabs 22 using the opposite side.

Again, an alternative nonabsorbent solid plastic, woven. plastic braid, or closed-cell foam material can be used to substitute for the closed-cell polymer foam pieces 24′ and 24″. Also one of several different fasteners can be used to connect the medical tube holder to the flange 52 to the medical tube 50.

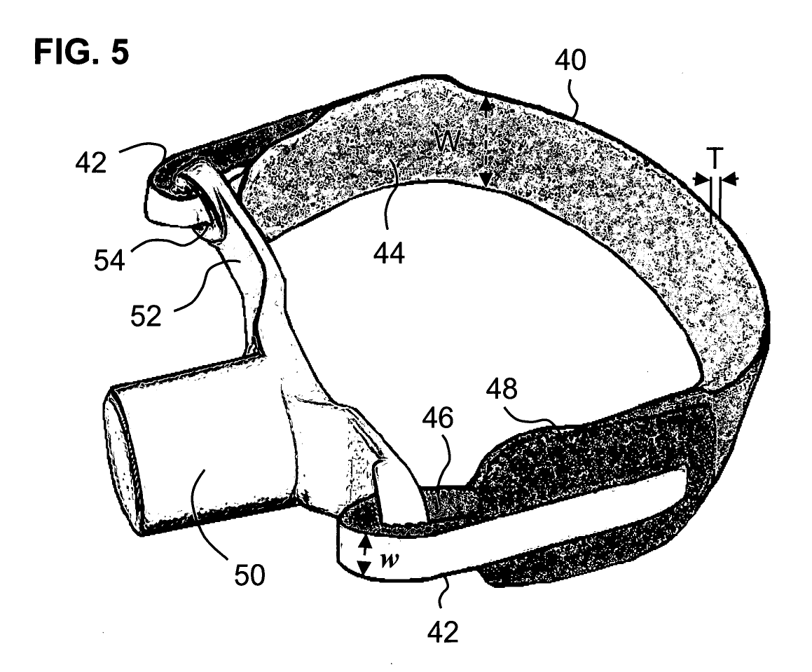

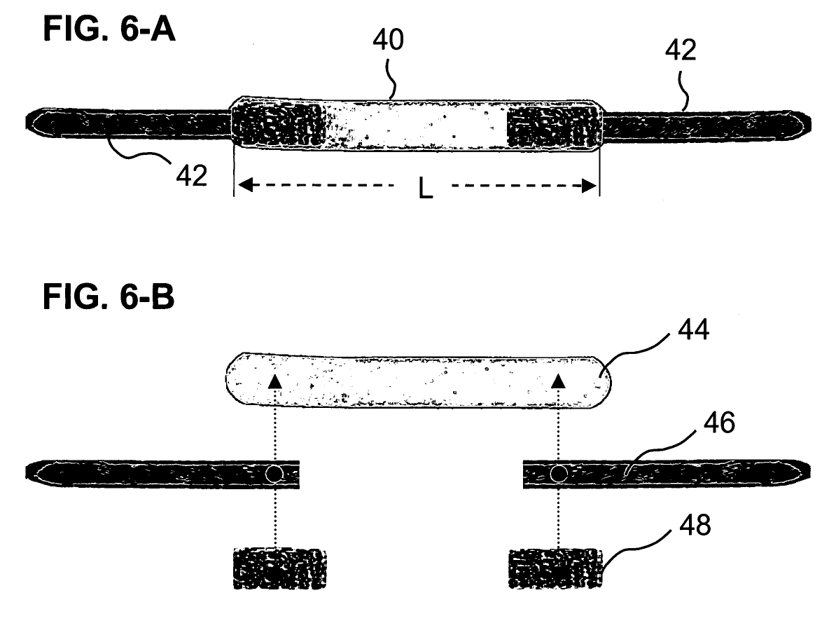

An alternative embodiment of the present invention is illustrated in FIG. 5 (perspective view), FIG. 6-A (side view), and FIG. 6-B (side view). The medical tube holder is constructed in a manner similar to that of the primary embodiment and includes a modified nonabsorbent retention band 40 with two fastener tabs 42 wrapped through the slots 54 of a flange 52 of a medical tube 50.

The physical dimensions (W, w, T, L) of the alternative embodiment are sized similarly to those of the primary embodiment. Also similar to the primary embodiment, the fastener tabs 42 are up to fifty percent of the length L of the nonabsorbent retention band 40. The fastener tabs 42 wrap back and attach firmly to two loop fastener pieces 48 that are coupled to the nonabsorbent retention band 40. Any portion of the fastener tabs 42 that extend past the two loop fastener pieces 48 can be removed with a safety scissors.

A neoprene piece 44 that is situated closest to the patient is used to provide comfort and prevent the absorption of moisture. A hook fastener piece 46 is covered with a loop fastener piece 48, and the combination is secured to each end of the neoprene piece 44 using a series of heavy stitches. The loop fastener piece 48 is much wider than the hook fastener piece 46, but is set in from the edges of the neoprene piece 44.

The medical tube holder defined in this alternative embodiment does not require the use of neoprene. Any of several flexible, nonabsorbent materials can be used to provide patient comfort, prevent the absorption of moisture, hold the medical tube 50 securely, and remain hygienic.

For all embodiments the combination of the nonabsorbent material and the custom or adjusted length of the retention band provides a safe, comfortable fit and allows a patient to wash or be washed with the device left in place. Also, if a medical tube does need to be changed, the medical tube holder is easy to get on and off.

Although the present invention has been described in detail, those skilled in the pertinent art should understand that they can make various changes, substitutions and alterations herein without departing from the spirit and scope of the invention in its broadest form.

What is claimed is:

1. A medical tube holder, comprising:
a band of nonabsorbent material coupled to a first fastener protruding from said band from a first and a second end; and
a second fastener coupled to said first fastener; whereby the protrusions from said first fastener can loop back and attach to said second fastener.

2. The medical tube holder as recited in claim 1 wherein said nonabsorbent material is selected from a group comprising solid plastic, woven plastic, closed-cell polymer foam, rubber, neoprene, polypropylene, polyethylene, polyvinyl chloride, and nylon.

3. The medical tube holder as recited in claim 1 wherein said band has a length based on a circumference of a patient's anchoring appendage.

4. The medical tube holder as recited in claim 3 wherein said first fastener has a length approximately twice said length of said band.

5. The medical tube holder as recited in claim 3 wherein said second fastener has a length approximately equal to said length of said band.

6. The medical tube holder as recited in claim 1 wherein said second fastener has fastening material on two sides.

7. The medical tube holder as recited in claim 1 wherein said fasteners are selected from a group comprising: an adhesive material, a hook and loop material, a mushroom stem material, and a tongue and groove material.

8. A medical tube holder, comprising:
a first band of nonabsorbent material coupled to a first fastener protruding from said first band from a first end;
a second band of nonabsorbent material coupled to a second fastener protruding from said second band from a first end;
a third fastener coupled to said second band at a second end, said third fastener couplable to a portion of said first fastener of said first band; and
a fourth fastener coupled to a portion of said first fastener of said first band and coupled to said second fastener of said second band; whereby the protrusions from said first fastener and said second fastener can loop back and attach to said fourth fastener.

9. The medical tube holder as recited in claim 8 wherein said nonabsorbent material is selected from a group comprising solid plastic, woven plastic, closed-cell polymer foam, rubber, neoprene, polypropylene, polyethylene, polyvinyl chloride, and nylon.

10. The medical tube holder as recited in claim 8 wherein said second band has a length substantially shorter than a circumference of a patient's anchoring appendage.

11. The medical tube holder as recited in claim 8 wherein said first band has a length approximately longer than a circumference of a patient's anchoring appendage.

12. The medical tube holder as recited in claim 8 wherein said fourth fastener has a length approximately equal to a combined length of said first band coupled to said second band.

13. The medical tube holder as recited in claim 8 wherein said fourth fastener has fastening material on two sides.

14. The medical tube holder as recited in claim 8 wherein said fasteners are selected from a group comprising: an adhesive material, a hook and loop material, a mushroom stem material, and a tongue and groove material.

15. A medical tube holder, comprising:
a band of nonabsorbent material coupled to a first fastener and a second fastener protruding from said band from a first end and a second end; and
a third fastener and a fourth fastener coupled to said band at said first end and said second end; whereby the protrusions from said first fastener and said second fastener can loop back and attach to said third fastener and said fourth fastener.

16. The medical tube holder as recited in claim 15 wherein said nonabsorbent material is selected from a group comprising solid plastic, woven plastic, closed-cell polymer foam, rubber, neoprene, polypropylene, polyethylene, polyvinyl chloride, and nylon.

17. The medical tube holder as recited in claim 15 wherein said band has a length based on a circumference of a patient's anchoring appendage.

18. The medical tube holder as recited in claim 17 wherein said first fastener and said second fastener have lengths approximately one half of said length of said band.

19. The medical tube holder as recited in claim 15 wherein said third fastener and said fourth fastener have lengths of approximately one inch.

20. The medical tube holder as recited in claim 15 wherein said fasteners are selected from a group comprising: an adhesive material, a hook and loop material, a mushroom stem material, and a tongue and groove material.
